(19) United States
(12) Patent Application Publication
(10) Pub. No.: US 2009/0082618 A1
(43) Pub. Date: Mar. 26, 2009

(54) Device for the prevention of urinary incontinence in humans

(76) Inventors: Wolfgang Abele, Tuttlingen/Donau (DE); Erich K. Odermatt, Schaffhausen (CH)

Correspondence Address: THE NATH LAW GROUP, 112 South West Street, Alexandria, VA, 22314, US

(21) Appl. No.: 11/919,825

(22) PCT Filed: May 4, 2006
(86) PCT No.: PCT/EP2006/004177

§ 371 Date: Jan. 16, 2008

(30) Foreign Application Priority Data
May 4, 2005 (DE) 10 2005 021 893.8

(51) Int. Cl. A61F 2/04

(52) U.S. Cl. 600/31

(57) ABSTRACT

The invention relates to a device (1) for the prevention of urinary incontinence in humans, with an elongate expandable balloon (8) made of elastic material and with a flat band (2) which extends in the longitudinal direction of the balloon, is made of textile material, and is connected to the balloon (8) in a planar manner. In the area of the balloon, the band (2) of textile material is embedded in the elastic material of the balloon on all sides, and its band sections (3, 16) protruding from both ends of the embedding are free from a coating.

The invention relates to a device for the prevention of urinary incontinence in humans, with an elongate expandable balloon made of elastic material and with a flat band which extends in the longitudinal direction of the balloon, is made of textile material, and is connected to the balloon in a planar manner.

Urinary incontinence bands with a balloon made of elastic material are known. Reference is made in this connection to DE 43 28 158 A1, WO 00/18319, WO 00/66030 and WO 03/013392.

In the incontinence band according to WO 00/18319, a band made of nylon fibers and impregnated with silicone is provided. An expandable balloon is connected to a surface of the band. Silicone-impregnated bands of fibers pose certain problems with regard to fixing. By contrast, open-pore textile bands have proven more suitable.

The object of the invention is to make available a urinary incontinence band which is easy to produce and permits safe handling during implantation.

The invention is characterized in that, in the area of the balloon, the band of textile material is embedded in the elastic material of the balloon on all sides, and its band sections protruding from both ends of the embedding are preferably free from a coating.

A good join between band and balloon is created by the invention. The production of the band is also greatly simplified. Thus, on the one hand, the balloon can be designed as an open shell. On the other hand, the band section to be connected to the balloon can be embedded separately into the elastic material of the balloon. This band section can then be used as a cover for the half-shell of the balloon and can be connected to the half-shell of the balloon by adhesive bonding, as a result of which a balloon is formed which is reinforced on one side, namely on the band side.

In one embodiment of the invention, the balloon has the shape of a flat hollow rectangular parallelepiped. According to the invention, a balloon with a thin elastically extensible wall on one side and with a thicker wall on the other side can be obtained, wherein the band is embedded in the thicker wall and stabilizes it. The textile band is preferably a knitted band. It is preferably composed of monofilament threads, in particular ones made of polypropylene. The longitudinal edges of the band can be tied off at least in sections adjoining the balloon. If no special securing means are provided for securing the band, then the longitudinal edges in the area of the band ends are preferably not tied off, i.e. there are free ends of the monofilament threads here that can hook in the connective tissue.

The embedding of the band preferably protrudes beyond the ends of the balloon, in particular in the form of elastic extensions that protrude beyond both ends of the balloon. The connection between band and balloon is stabilized in this way. Moreover, twisting of the band in this area is avoided during implantation. Generally, it is sufficient for these extensions to have a length of the order of the band width, i.e. ca. 10 mm.

The band itself is preferably designed as an open-pore lattice. The clear lattice apertures are preferably in the range of 3 to 5 mm. This permits a rapid incorporation of the band. The band is preferably nonextensible or only slightly extensible. In this way, the balloon on the band side is also nonextensible or only slightly extensible and also does not bulge out. The pressure exerted by the balloon can thus be applied in a targeted manner in one direction. The wall of the balloon can vary in thickness, in order to achieve a preferred initial extension at the thinner areas, in particular in the central area.

The balloon is made of soft elastic material, in particular of silicone. The soft elastic material can be transparent, in particular colorless. It is also possible for X-ray contrast media, or other substances that can be detected by physical methods, to be integrated into the soft elastic material so as to be able to monitor the position of the balloon. In this case, the balloon is opaque. In one embodiment of the invention, the balloon interior is connected in a manner known per se to a flexible fluid conduit. The latter preferably opens at one end of the balloon into a narrow longitudinal side of the balloon. The connection is advantageously an adhesive connection. As has already been mentioned above, securing means for fixing the band in the body can be provided at the band end. Fixing can be achieved, for example, by screwing the ends of the band onto bone, in which connection it should be noted, however, that patients with urinary incontinence are older and often suffer from osteoporosis, which makes fixation by screwing to bone unsuitable. For osteoporosis patients in particular, it is preferable for the ends of the band to be designed in a loop shape, in particular as textile adhesive fasteners, which can be placed round the bone in the pelvic region and closed. Securing is also possible by self-adhesion if the band has a rough structure. It is also conceivable that the rough structure of the band is self-adhesive and holds the device in position in the body without a loop-shaped connection. It is alternatively possible to use an adhesive to temporarily fix the band until the latter has become incorporated. The implant can be implanted using a minimally invasive operating procedure analogous to the transobturator technique (TOT) in female urinary incontinence or by another minimally invasive operating technique from the field of female urinary incontinence. Other alternative access routes from below are also possible, for example access from the region of the perineum or the region of the os pubis, for placing the implant by a minimally invasive route. The implant can also be placed above or below the urethra, depending on which minimally invasive access route is chosen. Placement of the balloon above the urethra is the preferred implantation technique.

In a very particularly preferred embodiment of the invention, the balloon is provided, at least on one wall side, with a soft and/or porous padding material. This is normally the side facing the urethra after implantation. It is in particular the thin side that can bulge up and is directed away from the side strengthened by the band. This embodiment with the padding material can also be used independently of the other features of the invention and can generally be provided in balloons which, after implantation, come into contact with the urethra. It has in fact been found that smooth balloon walls, particularly those made of silicone, do not form a join with the connective tissue after implantation, in particular not with the tissue of the urethra. This can lead to relative movements between the balloon and the urethra, which can result in the wall material of the urethra being irritated or even eroded. This situation can be remedied by providing the wall material of the balloon with a soft and/or porous padding into which cells are able to grow. In this way, a join can be created between the urethra and the surface of the balloon, and irritation or erosion of the wall of the urethra can be avoided. The padding material can be of natural origin or synthetic. It can be absorbable or nonabsorbable. The pore size of the padding material is generally over 20 μm and in particular is 100 μm or more. Cells can grow very effectively into such pores and thus form an anchoring for the connective tissue. Soft padding material is preferably structured, such that the tissue cells find anchoring sites. The mechanical irritation caused by the implantation results in the formation of scar tissue, which represents a strengthening of the wall of the urethra in this area. Even if the padding material is absorbable, the scar tissue is preserved after the absorption and affords effective protection of the urethra.

Suitable padding materials are those of a textile nature, such as wovens, drawn-loop knits, braids and nonwovens. Of these, elastic materials are preferred such as formed-loop knits, and in particular nonwovens. An example of a synthetic nonwoven is a nonwoven composed of linear polyurethane, which is obtained by spray-coating. Examples of absorbable nonwovens made from biological material are ones from biological pericardial tissue and nonwovens made of collagen from bovine hide. The nonwovens and the other padding materials can be connected to the balloon by adhesive bonding or can be fixed thereto. Mechanical means are also suitable. Thus, it is also possible to completely encapsulate the balloon or to receive it in a tube section made of the padding material. It is not necessary for the padding material to be connected fixedly or permanently to the balloon. A temporary connection or local mechanical fixing is sufficient and may be preferred. After the cells have grown into the porous padding material, the latter is more part of the connective tissue than of the incontinence band. It can therefore be preferable to design the padding material such that it has no connection to the balloon after incorporation of the implant. Thus, any adhesive used, or the entire padding material, can be made absorbable.

The materials of the urinary incontinence band according to the invention can also be provided in a manner known per se with active substances, in particular with antimicrobial agents. These can be provided at least on the outer surface of the materials or can be integrated into the material. The padding material too can accordingly be provided with antimicrobial agents. It is also possible to provide the surface of the balloon with substances that promote wound healing. The elastic material can be pretreated such that ionic and/or covalent bonds with the active substances are possible.

The device according to the invention can be implanted both in male and female patients, the balloon preferably being placed above the urethra in male patients and preferably below the urethra in female patients. The balloon can be bent in a U-shaped manner in the longitudinal direction, such that it partially encloses the urethra.

Further features of the invention will become clear from the following description of preferred embodiments in conjunction with the drawing and with the dependent claims. Here, the individual features can each be realized either singly or severally in an embodiment of the invention.

In the drawing:

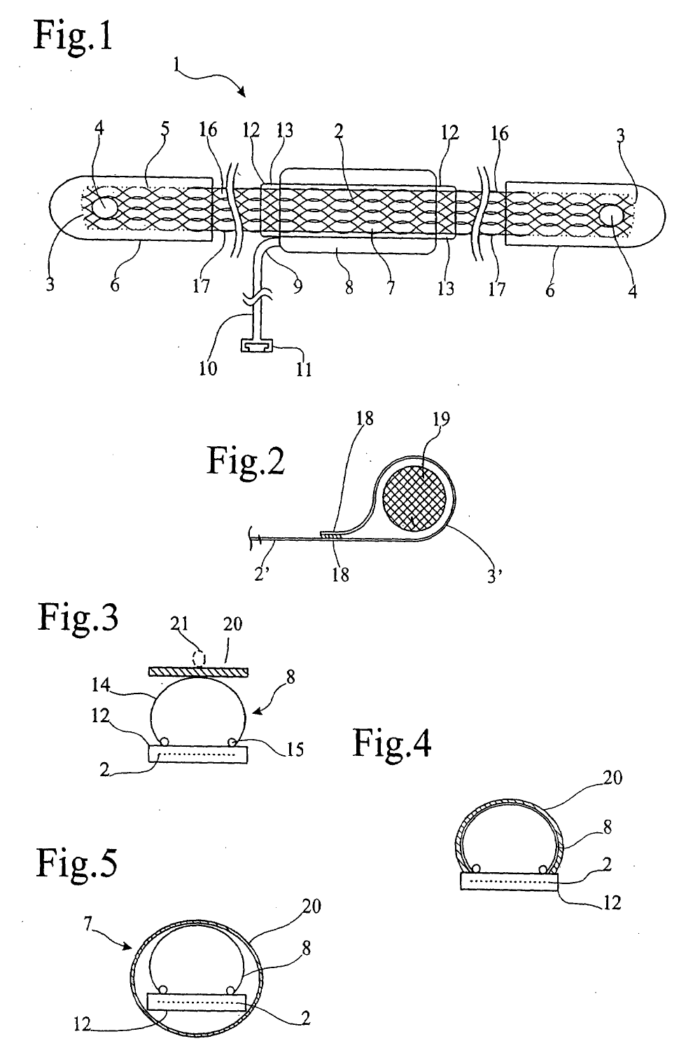

FIG. 1 shows a plan view of a device according to the invention,

FIG. 2 shows an alternative design of fixing elements,

FIGS. 3-5 show alternative embodiments of the devices according to the invention in cross section and schematically, the expandable balloon being provided with a padding material.

In the embodiment of the invention shown in FIG. 1, a device 1 for the prevention of urinary incontinence in humans comprises a band 2, which is designed as a formed-loop knit monofilament band of polypropylene and has a mesh-like structure with a clear mesh width of ca. 1 mm. The band 2 has a length of ca. 50 cm. At its ends 3, the band has two openings 4 for securing it to a needle (not shown), with the aid of which the band can be implanted. In the area of the ends 3, the band 2 has free ends 5 of the monofilament thread lying at its margins. These free ends 5 serve to engage the band in the human tissue, in order to ensure a good anchoring of the band ends. To protect the biological tissue when introducing the band, the band ends 3 are enclosed by polyethylene sleeves 6. These cover the free ends 5 of the thread and also have corresponding openings 4. In the area of the openings 4, the sleeves can be welded to the band 2 in order to strengthen the band. In the central area 7 of the band, the band is provided with a balloon 8, which is secured on the band 2. The balloon 8 is made of soft elastic silicone. It is designed roughly as a flat rectangular hollow body. A fluid conduit 10, which is connected sealingly to the balloon wall, opens into the balloon 8 on a short narrow side 9 thereof. The fluid conduit 10 is composed of a silicone tube and, at its free end, has a fluid valve 11 equipped with a permanently elastic septum (not shown) through which fluid can be introduced into the balloon or withdrawn from it. The balloon geometry can be configured such that, when the fluid is injected, the central part of the balloon is first extended. This can generally be obtained by a variation of the balloon wall thickness, in particular by a thickening of the balloon material at the ends. The balloon can also already have a curved shape from the production process.

In the central area 7, the band 2 has a reinforcement 12 which is likewise made of silicone and encloses the band in this area on all sides. The balloon 8 is adhesively affixed to this reinforcement 12, and extensions 13 of the reinforcement protrude beyond the balloon 8 on both longitudinal sides of the balloon.

The reinforcement 12, into which the band 2 is embedded, forms a wall of the balloon. It is therefore possible to first design the balloon as an open shell 14 (FIG. 3) which is placed with downwardly facing edges 15 onto the reinforcement 12 and is adhesively affixed to the latter in a sealed manner. The cross section of the band in the central area 7 is shown schematically in FIGS. 3 to 5. Intermediate areas 16 of the band between the ends 3 and the central area 7 have edges 17 into which the monofilament thread is bound, i.e. there are no protruding free thread ends. In these intermediate areas, the band does not have to be provided with a sleeve, since its smooth margins mean that it can be drawn through the connective tissue without damage. Instead of the band ends with the free thread ends on the margin of the band, the band can also be provided at its ends with other securing means for fixing it in the body. Thus, the band 2′, as shown schematically in FIG. 2, can be provided at its ends 3′ with textile adhesive fasteners 18, which can be placed around a bone 19.

In the embodiments according to FIGS. 2 to 4, the surface of the balloon 8 directed away from the band 8 is provided with a porous protective layer 20 for protecting the urethra 21 (indicated by broken lines). In the embodiment shown in FIG. 3, the protective layer is composed of a substantially flat strip 20 of soft padding material that is connected to the balloon permanently or releasably. After the implantation, the protective layer induces the connective tissue in the area of the urethra to form new cells, which penetrate into the porous material of the protective layer and connect firmly thereto. This newly formed tissue creates, together with the protective layer 20, a permanent intermediate layer between balloon and urethra and protects the urethra from mechanical damage by the material of the balloon 8.

As is shown in FIG. 4, the protective layer 20 can substantially enclose the balloon. In the embodiment according to FIG. 5, the whole central area 7 of the device according to the invention is encapsulated by the protective layer, which, for example, can have a tubular configuration. The protective layer 20 is preferably designed as a porous nonwoven. It can be made of synthetic material, such as linear polyurethane, which is not absorbable. However, it can also be made of absorbable material, such as collagen, which has performed its function after the formation of the newly formed connective tissue, since the newly formed connective tissue takes over the protective function.

The embodiments according to FIGS. 3 to 5 can also be realized independently of the embodiment according to FIG. 1 and independently of the band reinforcement, since the soft and/or porous padding provided on the balloon side directed toward the urethra can also perform its functions independently of the design of the rest of a device for the prevention of urinary incontinence.

1. A device (1) for the prevention of urinary incontinence in humans, with an elongate expandable balloon (8) made of elastic material and with a flat band (2) which extends in the longitudinal direction of the balloon, is made of textile material, and is connected to the balloon (8) in a planar manner, wherein, in the area of the balloon, the band (2) of textile material is embedded in the elastic material of the balloon on all sides.

2. The device as claimed in claim 1, wherein the balloon (8) has roughly the shape of a flat hollow rectangular parallelepiped, with a thin and elastically extensible wall on one side and with a thicker wall (12) on the other side, and wherein the band (2) is embedded in the thicker wall.

3. The device as claimed in claim 1, wherein the band (2) is a formed-loop knit band (2) of monofilament threads.

4. The device as claimed in claim 1, wherein the embedding of the band (2) protrudes in the form of elastic extensions (13) beyond both ends of the balloon (8).

5. The device as claimed in claim 4, wherein the length of the extensions (13) is of the order of the width of the band.

6. The device as claimed in claim 1, wherein the band (2) is designed as an open-pore lattice.

7. The device as claimed in claim 1, wherein a thicker wall (12) of the balloon is not extensible in the longitudinal direction by the band (2).

8. The device as claimed in claim 1, wherein the elastic material of the balloon (8) is transparent.

9. The device as claimed in claim 1, wherein the balloon interior is connected to a flexible fluid conduit (10), which opens at one end of the balloon into a narrow longitudinal side (9) of the balloon.

10. The device as claimed in claim 1, wherein the band (2) has securing means (4; 18) at its free ends.

11. The device as claimed in claim 1, wherein the band (2) has loop-shaped textile adhesive fasteners (18) at its ends (3).

12. A device for the prevention of urinary incontinence in humans, with an expandable balloon made of elastic material and with a band connected to the balloon, in particular as claimed in claim 1, wherein the balloon (8), at least on one wall side intended to bear on the urethra, is provided with a soft and/or porous padding material (20) into which cells are able to grow.

13. The device as claimed in claim 12, wherein the padding material (20) is soft and porous.

14. The device as claimed in claim 12, wherein the padding material is absorbable.

15. The device as claimed in claim 1, wherein at least the outer surface of the materials is provided with active substances.

16. The device as claimed in claim 1, wherein substances that promote wound healing are provided on the surface of the balloon.

17. The device as claimed in claim 1, wherein the elastic material of the balloon has, on its surface, reactive sites that are able to form ionic and/or covalent bonds with active substances.

18. A device for the prevention of urinary incontinence in humans, with an expandable balloon made of elastic material and with a band connected to the balloon, in particular as claimed in claim 9, wherein the balloon (8), at least on one wall side intended to bear on the urethra, is provided with a soft and/or porous padding material (20) into which cells are able to grow.

19. The device as claimed in claim 13, wherein the padding material is absorbable.

20. The device as claimed in claim 16, wherein the elastic material of the balloon has, on its surface, reactive sites that are able to form ionic and/or covalent bonds with active substances.

21. The device as claimed in claim 1, wherein the elastic material of the balloon (8) is colorless.

22. The device as claimed in claim 1, wherein at least the outer surface of the materials is provided with an antimicrobial agent.
